(19) United States
(12) Patent Application Publication
(10) Pub. No.: US 2015/0257701 A1
(43) Pub. Date: Sep. 17, 2015

(54) BODY FAT DIAGNOSTIC APPARATUS

(71) Applicants: OSAKA PREFECTURE UNIVERSITY PUBLIC CORPORATION, Osaka (JP); PUBLIC UNIVERSITY CORPORATION OSAKA CITY UNIVERSITY, Osaka (JP); ADVANTEST CORPORATION, Tokyo (JP)

(72) Inventors: Hiromichi HORINAKA, Osaka (JP); Hiroyasu MORIKAWA, Osaka (JP)

(21) Appl. No.: 14/632,906

(22) Filed: Feb. 26, 2015

(30) Foreign Application Priority Data
Mar. 14, 2014 (JP) 2014-052620

(51) Int. Cl. A61B 5/00; A61B 8/14; A61B 8/08

(52) U.S. Cl. CPC A61B 5/4872; A61B 8/5223; A61B 8/5207; A61B 8/14; A61B 8/5246; A61B 8/08

(57) ABSTRACT

A body fat diagnostic apparatus, with which a change in the velocity of ultrasonic waves can be measured and fat can be diagnosed while suppressing the effects of a periodical change due to respiration and heartbeats of a subject, is formed of: a process unit for sequentially acquiring ultrasonic wave echo signals for a number of frames so as to form a group of B mode images; a data storage unit for storing the ultrasonic wave echo signals corresponding to the groups of B mode images; a sampling unit for sampling one frame as a standard image and another frame as a comparative image through the calculation of a cross correlation; and a velocity change analyzing unit for forming an image by calculating a change in the velocity of ultrasonic waves on the basis of the ultrasonic wave echo signals corresponding to the standard image and the comparative image.

### CROSS-REFERENCE TO RELATED APPLICATIONS

This application claims priority to Japanese Patent Application No. 2014-052620 filed Mar. 14, 2014, the subject matter of which is incorporated herein by reference in entirety.

### BACKGROUND OF THE INVENTION

1. Field of the Invention

The present invention relates to a body fat diagnostic apparatus for applying heat to a measurement region such as a region of interest (ROI) of a subject so as to detect a change in the velocity of ultrasonic waves before and after the application of heat to the region, and thus, for diagnosing fat tissues. In particular, the present invention relates to a body fat diagnostic apparatus that is appropriate for fat diagnosis in a living body where periodical changes take place due to physiological movements such as of respiration and heartbeats.

2. Description of Related Art

As a diagnostic technology for diagnosing the state within the body, a sound wave measuring apparatus for applying heat to a subject through irradiation of energy so as to measure a change in the velocity of ultrasonic waves before and after the application of heat, and thus, for measuring the characteristics of the temperature change or the energy absorbing properties in the heated portion has been disclosed (see Patent Document 1).

In addition, a method and an apparatus for detecting fat tissues in order to diagnose the distribution of fat tissues, wherein heat is applied to a region of interest through irradiation of light, and a change in the velocity of ultrasonic waves before and after the application of heat is measured so as to detect and define a portion where a change in the velocity of ultrasonic waves has a negative value as fat tissues in order to diagnose visceral fat which is one risk factor of lifestyle related diseases, have been proposed as a new technique for image diagnosis using a change in the velocity of the ultrasonic waves before and after the application of heat (Patent Document 2).

The body fat diagnostic apparatus (body fat tissue detecting apparatus) in Patent Document 2 is described below. This apparatus is provided with the main body of the apparatus equipped with a control unit that is necessary to acquire a B mode cross sectional image or an image showing a change in the velocity of ultrasonic waves and a probe that is made to make direct contact with the body surface of a subject so that heat can be applied to the subject or the subject can be irradiated with ultrasonic waves. This probe is a dedicated probe made up of a linear array probe for irradiating the measurement region of a subject with ultrasonic waves and an infrared ray laser source located next to the linear array probe for irradiating the measurement region of the subject with near infrared rays in order to apply heat, where the two are placed side by side laterally so as to target the same measurement region.

The linear array probe has a number of oscillators (formed of piezoelectric elements) that are linearly aligned. Each oscillator sends an ultrasonic wave signal when pulse waves are excited by a drive signal from the control unit, and receives an ultrasonic wave echo signal from the inside of the body of the subject in response to this ultrasonic wave signal. In addition, scanning is made possible by sequentially switching the oscillators for wave transmission and reception in response to a control signal. Furthermore, the infrared ray laser source allows the emission of near infrared rays having a wavelength of 700 nm to 1000 nm from a side of the linear array probe.

The operation of this apparatus for measuring fat in which a change in the velocity of ultrasonic waves is measured is described below. A subject is irradiated with near infrared rays from the infrared laser source so that heat is applied to the subject, and after a predetermined period of time has elapsed, a linear array probe is driven so as to send an ultrasonic wave signal in pulse form for sequential scans, and at the same time, ultrasonic wave echo signals, which are signals received from the subject are sequentially received. In addition, the waveform of the ultrasonic wave signals (reception signals) acquired in a state that the subject is irradiated with the rays is stored as an ultrasonic echo signal after irradiation with rays.

Irradiation with rays is stopped when the storing of the waveform of the received ultrasonic wave signals is completed after irradiation with rays. When a predetermined period of time has elapsed after the stoppage of irradiation and the temperature of the subject has lowered sufficiently, the linear array probe is driven so as to send an ultrasonic wave signal, and the same time, an ultrasonic wave echo signal is received from the subject. Thus, the waveform of the ultrasonic wave signal (reception signal) acquired in a state that irradiation with rays is stopped is stored as an ultrasonic wave echo signal at the time of no irradiation. Here, the stored ultrasonic wave echo signals are displayed as a B mode cross sectional image when the amplitudes thereof are displayed as the brightness.

Next, a change in the velocity in the ultrasonic waves is found from the below described relationship on the basis of the ultrasonic wave echo signals after irradiation with rays and at the time of no irradiation.

FIG. 9 is a schematic diagram showing ultrasonic wave signals at the time of no irradiation (before the application of heat) and ultrasonic wave signals after irradiation with rays (after the application of heat) during a certain part of section. The velocity of the ultrasonic waves at the time of no irradiation is V, and the velocity of the ultrasonic waves after irradiation with rays is V′. In addition, the pulse interval that occurs when the ultrasonic wave signals propagate through a section between certain borders at the time of no irradiation is τ, and the pulse interval that occurs when the ultrasonic wave signals propagate across the same section between the borders (same distance) after irradiation with rays is τ−Δτ. That is to say the change in the temperature has made the pulse interval shorter by Δτ. Here, the following relationship in Formula (1) is achieved, and thus, a change in the velocity of the ultrasonic waves can be calculated from Formula (2) in the following on the basis of the chronological change in the pulse interval between the two echo signals.

V·τ=V′·(τ−Δτ) (1)

V′/V=τ/(τ−Δτ) (2)

Accordingly, the pulse interval (τ) in the region of interest and the amount of the sift in the waveform (Δτ) are calculated from the two echo signals that have been measured, and the change in the velocity of the ultrasonic waves (ratio of the change in the velocity of the ultrasonic waves (V′/V)) in each portion is calculated from Formula (2).

Next, the portions where the calculated ratio of the change in the velocity of the ultrasonic waves (V′/V) has a value smaller than 1 (regions where the ratio of the change in the velocity of the ultrasonic waves after the application of heat is negative) are determined as a fat region from among the portions where the ratio of the change in the velocity of the ultrasonic waves has been calculated.

That is to say, the velocity of ultrasonic waves that propagate through water is 1525 msec, and the velocity of ultrasonic waves that propagate through fat is 1412 msec at 37° C. The changes in the velocity of the ultrasonic waves propagating through the two media when the temperature changes are as follows:

Water: +2 m/sec·° C.

Fat: −4 msec·° C.

Therefore, the velocity of the ultrasonic waves the velocity increases as the temperature of the muscles and organs (liver and the like) that contain a large amount of water increases, but the velocity of the ultrasonic waves decreases in fat portions. Thus, the polarity of the change in the velocity of the ultrasonic waves is opposite for these two different media.

Thus, a fat region can be detected by specifying a region where the change in the velocity of the ultrasonic waves has a negative value when the temperature of the measurement region is increased.

In addition, the distribution of the change in the velocity of the ultrasonic waves resulting from the analysis is imaged and displayed on a display unit, and thus, fat regions can be clearly distinguished from the other portions in the image.

### PRIOR ART DOCUMENTS

### Patent Documents

Patent Document 1: Japanese Unexamined Patent Publication 2001-145628

Patent Document 2: Japanese Unexamined Patent Publication 2010-005271

### SUMMARY OF THE INVENTION

### 1. Problem to be Solved by the Invention

The body fat diagnostic apparatus in Patent Document 2 can display an image of a fat region by applying heat to the measurement region and by measuring a change in the velocity of the ultrasonic waves using a dedicated probe having a linear array probe and an infrared ray laser source that are aligned side by side.

However, a problem arises as described below in the case where fat is diagnosed using an ultrasonic wave velocity change image in a living body.

In order to acquire an ultrasonic wave velocity change image, ultrasonic wave echo signals having a number of (for example 128) scans that are required for the B mode image formation are acquired for different temperatures before and after the application of heat, respectively, and then, the arithmetic operation of Formula (2) is carried out on the basis of the corresponding sections of portions of the ultrasonic wave echo signals in the acquired B mode images before and after the application of heat.

In living bodies, periodical changes of the borders between tissues such as dilation, contraction and vibration take place due to physiological movements such as of respiration and heartbeats. Therefore, in the case where an ultrasonic wave echo signal having a number of scans required for the formation of a B mode image is acquired using a probe, a periodic change may affect the formation of the ultrasonic wave velocity change image depending on the timing for acquiring an ultrasonic wave echo signal during the period of the periodic change, which causes an error in measurement or ambiguity.

As for the effects of respiration, in some cases, the subject should be asked to hold breath temporarily so that the image quality can be improved. However, it may be difficult to ask some subjects such as suffering patients and infants to hold breath during the measurement. It is also difficult to ask the subject to hold breath in the case where the subject is an animal. Meanwhile, it is difficult to deliberately stop the heartbeat.

Therefore, an object of the invention is to provide a body fat diagnostic apparatus with which a change in the velocity of the ultrasonic waves can be measured and fat can be diagnosed by suppressing the effects of a periodical change due to respiration and heartbeats of a subject even when such a periodical change is taking place when ultrasonic wave echo signals that are required for fat diagnosis using a change in the velocity of ultrasonic waves are acquired at the two points of time before and after the application of heat.

### 2. Means for Solving Problem

In order to achieve the above described object, the present invention provides a body fat diagnostic apparatus for diagnosing fat through the formation of an ultrasonic wave velocity change image by forming a B mode image on the basis an ultrasonic wave echo signal having a number of scans for one frame that have been acquired from a measurement region and at the same time by calculating a change in the velocity of ultrasonic waves on the basis of ultrasonic wave echo signals having a number of scans for one frame that have been acquired from the measurement region before and after the application of heat, respectively, with: a process unit for forming a group of B mode images of a number of frames within one period during the periodical change by sequentially acquiring ultrasonic wave echo signals having a number of scans that is required for the formation of a the above described group of B mode images for a number of frames from a measurement region that changes periodically; a data storage unit for storing ultrasonic wave echo signals that correspond to the group of the B mode images for a number of frames that have been acquired before and after the application of heat respectively; a sampling unit for sampling one frame as a standard image through the calculation of the cross correlation that indicates the degree of a change in the image between frames in either group of B mode images before or after the application of heat and for sampling one frame as comparative image through the calculation of the cross correlation that indicates the degree of a change in the image between each frame of the other group of B mode images before or after the application of heat of the above described standard image; and an ultrasonic wave velocity change analysis unit for forming an ultrasonic wave velocity change image by calculating a change in the velocity of ultrasonic waves on the basis of an ultrasonic wave echo signal having a number of scans corresponding to the above described standard image and the above described comparative image.

In general, a periodic change resulting from the superposition of the movements of respiration and heartbeats takes place in living bodies. In the present invention, the process unit sequentially acquires an ultrasonic wave echo signal having a number of scans required for the formation of a group of B mode images for number of frames in order to form a group of B mode images for a number of frames corresponding to a number of points of time during one period of this periodic change. In addition, this process for sequentially acquiring an ultrasonic wave echo signal is carried out on the measurement region before and after the application of heat so as to form two groups of B mode images and at the same time so as to store the acquired ultrasonic wave echo signals in the data storage unit. The sampling unit samples one frame having a small change in the image between frames (image with high similarity) as a “standard image” by calculating the cross correlation between the frames in order to determine the frame from among the B mode images for a number of frames that are included in either one group of the stored B mode images before or after the application of heat. The cross correlation used for this arithmetic operation do not have a particular limitation as long as the arithmetic operation allows comparison of the degree of a change in the image (similarity) through the conversion to numeric values. The thus sampled “standard image” is a B mode image that has been acquired at the point of time when the subject is almost stationary because it has been selected when a change in the image due to the effect of the periodic change is small.

The sampling unit also samples one frame where the degree of change in the image is small (having high similarity) as compared to the sampled “standard image” from among the B mode images included in the group of the stored B mode images before or after the application of heat (B mode images in the group that is different from the group of B mode images including the “standard image”) by calculating the cross correlation in order to select the frame.

The thus sampled “comparative image” and the “standard image” are B mode images where the respiration and heartbeats are approximately in the same state, and in addition, B mode images at the point in time where respiration and heartbeat are almost stationary have been selected.

In addition, the ultrasonic wave velocity change analysis unit calculates the change in the velocity of ultrasonic waves on the basis of the ultrasonic wave echo signals that correspond to the selected “standard image” and “comparative image”, respectively, and forms an ultrasonic wave velocity change image. As a result, an ultrasonic wave velocity change image can be formed as a result of the calculation of a change in the velocity of ultrasonic waves from ultrasonic wave echo signals before and after the application of heat at a moment when respiration and heartbeats are in the same state and they are almost stationary.

### 3. Effects of the Invention

According to the present invention, an ultrasonic wave velocity change image can be formed on the basis of ultrasonic wave echo signals before and after the application of heat in accordance with such a timing that respiration and heartbeats are in the same state and at a moment when they are almost stationary, and therefore, fat diagnosis using the ultrasonic wave velocity change image that is least affected by respiration and by heartbeats can be made possible.

In the present invention, the number of scans per frame for forming a B mode image can be adjusted and the above described process unit may be formed so that the mode can be switched in such a manner that the scans per frame for forming a group of B mode images are thinned out and the number of frames of which the image can be taken during one period of the above described periodic change is increased.

When the scans thinned out and the number of scans is reduced, the frame rate can be increased and the number of frames of B mode image of which the image can be taken per unit hour can be increased. As a result, the standard image and the comparative image become more appropriate because they can be selected from among a greater number of frames. When scans per frame are thinned out, the resolution of the image deteriorates. However, the special change in the distribution of fat in the liver of the like is relatively small, and therefore, a very high resolution is not required. Therefore, the resolution is set high by increasing the number of scans per frame at the time of beforehand observation for initially determining the measurement position. After the measurement position has been determined, the scans are thinned out so as to increase the frame rate when the process unit acquires a B mode image, and thus, measurement that is appropriate for fat diagnosis becomes possible.

In the present invention, the cross correlation may be calculated using zero-mean normalized cross correlation (ZNCC) equation.

Here, ZNCC is given in Formula (3).

$$\begin{matrix}[ Formula 1 ] & \\ {R}_{ZNCC} = \frac{{\sum }_{j = 0}^{N - 1} {\sum }_{i = 0}^{M - 1} ( ( I ( i , j - \overline{I} ) ( T ( i , j ) - \overline{T} ) )}{\sqrt{{\sum }_{j = 0}^{N - 1} {\sum }_{i = 0}^{M - 1} {( I ( i , j ) - \overline{I} )}^{2} \times {\sum }_{j = 0}^{N - 1} {\sum }_{i = 0}^{M - 1} {( T ( i , j ) - \overline{T} )}^{2}}} & ( 3 )\end{matrix}$$

Here, the size of an image (number of pixels) is M×N, the amplitudes (brightness of the pixels) in the pixel location (i, j) in the image before and after the application of heat are I(i, j) and T(i, j) and the average amplitude (brightness of the pixels) in each image are as follows.

I̅, T̅ [Formula 2]

R_{ZNCC} has a value in a range of −1<R_{ZNCC}<1 and the closer to 1 the value is, the higher the cross correlation is.

The correlation where a change in the brightness between the corresponding pixels in the two images is generally represented by a numeric value can be obtained through the arithmetic operation using this formula, and thus, the degree of change in the image (similarity) can be represented by an appropriate numerical value.

In the present invention, a sum of zero-mean normalized cross correlation (ZNCC) may be used as the cross correlation for sampling the above described standard image.

This makes it possible to check the strength of the correlation between three or more adjacent frames, and therefore, images in a stationary state can further be represented by appropriate numerical values.

### DESCRIPTION OF THE DRAWINGS

FIG. 1 is a block diagram showing the entire configuration of the body fat diagnostic apparatus according to one embodiment of the present invention;

FIG. 2 is a diagram showing a portion of the configuration for sending and receiving an ultrasonic pulse wave signal for diagnosis and for sending ultrasonic continuous wave for applying heat using one probe;

FIG. 3 is a flow chart showing the procedure of the measurement operation for the body fat diagnostic apparatus;

FIG. 4 is a diagram illustrating arithmetic operation for the cross correlation in order to select a standard image;

FIG. 5 is graph showing an example of calculation for the sum SR(n) of cross correlations in an experiment on an animal;

FIG. 6 is a diagram illustrating an arithmetic operation for the cross correlation in order to select a comparative image;

FIG. 7 is graph showing an example of calculation for the cross correlation R_{ZNCC}(nS) in an experiment on an animal;

FIGS. 8A to 8C are images showing an example of an ultrasonic wave velocity change image; and

FIG. 9 is a schematic diagram showing ultrasonic wave echo signals before and after the application of heat.

### DETAILED DESCRIPTION OF EMBODIMENTS

(Configuration of Apparatus)

In the following the embodiments of the present invention are described in reference to the drawings. Here, a case where the liver (fatty liver) is measured is cited as an example for the description.

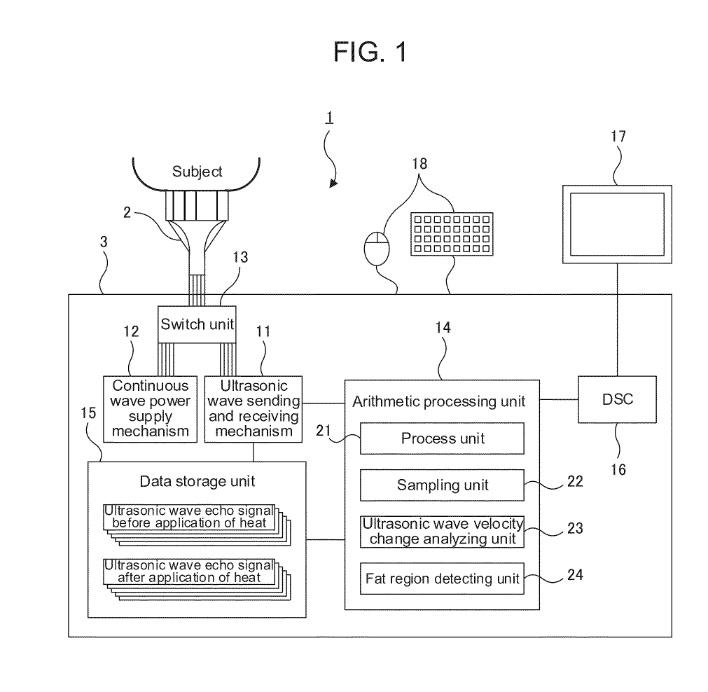

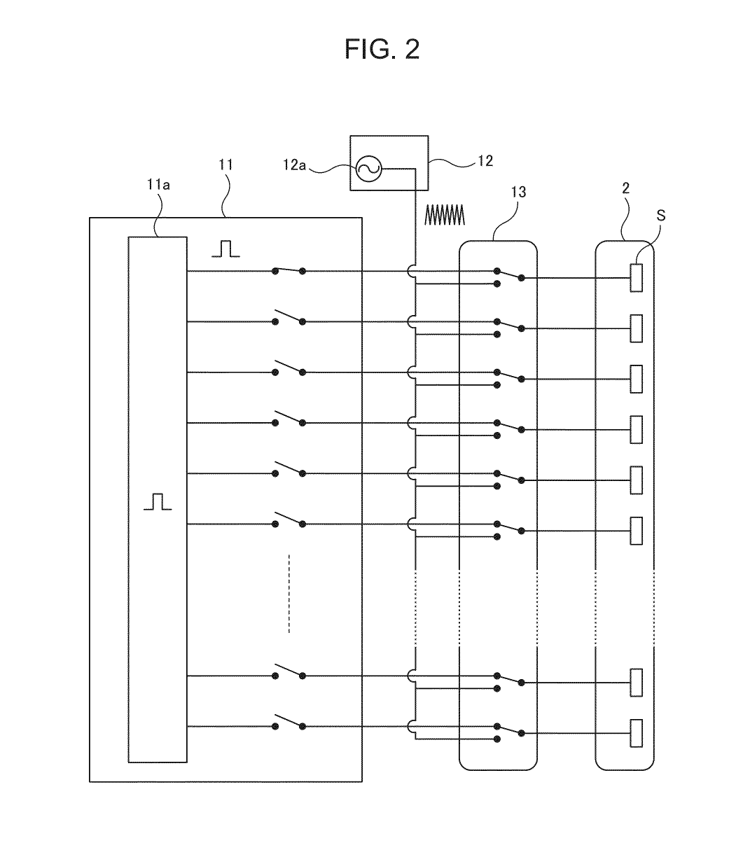

FIG. 1 is a block diagram showing the entire configuration of the body fat diagnostic apparatus according to one embodiment of the present invention, and FIG. 2 is a diagram showing a portion of the configuration for sending and receiving an ultrasonic pulse wave signal for diagnosis and for sending ultrasonic continuous wave for applying heat using one probe.

A body fat diagnostic apparatus 1 is formed of a probe 2 and a control unit 3 for controlling the system for ultrasonic wave diagnosis, for applying heat, for measuring a change in the velocity of ultrasonic waves and for fat diagnosis using the probe 2.

The probe 2 is an array type probe (also referred to as an array transducer) where a number of (for example 128) piezoelectric elements that function as oscillators for sending and receiving waves to and from a subject are aligned in a linear form. In order for the ultrasonic waves emitted from the oscillators to be able to reach the liver in a deep portion through adjacent ribs the thickness of the oscillators is smaller than the gap between the ribs, for example, 15 mm or less. Here, the conventional array type probes for an ultrasonic wave diagnostic apparatus that have been available in the market for the B mode image diagnosis can be used as the probe 2 without change as long as the thickness of the oscillators is appropriate.

The control unit 3 includes a computer having a memory, a CPU and an input/output apparatus, and generally controls the system that is required for the operation and analysis for a diagnosis using a B mode image and for a fat diagnosis. The components that relate to the present invention in this control can be divided into functional blocks for description, then is provided with an ultrasonic wave sending and receiving mechanism 11, a continuous wave power supplying mechanism 12, a switch unit 13, an arithmetic processing unit 14, a data storage unit (memory) 15, an image display control unit (digital scan convertor (DSC)) 16, a display unit (liquid crystal panel) 17 and an input unit (mouse, keyboard and the like) 18.

The ultrasonic wave sending and receiving mechanism 11 allows a drive circuit 11a to sequentially drive the system, and thus, controls the scanning by sending ultrasonic pulse waves from the probe 2 as an ultrasonic pulse wave signal for diagnosis so that the ultrasonic pulse waves excite the oscillators S in the probe 2 in a predetermined scanning order. The voltage of the pulse waves that have been sent is approximately 20 V to 60 V and the period of time during which the pulse lasts is approximately 0.5 μs to 5 μs.

Furthermore, the ultrasonic wave sending and receiving mechanism 11 controls the oscillators S so that each oscillator S stands by for and sequentially receives an ultrasonic wave echo signal that is reflected from the subject after an ultrasonic pulse wave signal has been sent. When such a control of sending and receiving a signal is repeated by the same number as that of the scans for one frame, a ultrasonic wave echo signal for forming a B mode image for one frame can be acquired.

The thus received ultrasonic wave echo signal for one frame is stored in the data storage unit (memory) 15, and at the same time, is sent to the arithmetic processing unit 14 so as to be read out for an arithmetic process whenever necessary.

In addition, the ultrasonic wave sending and receiving mechanism 11 can acquire the ultrasonic wave echo signal that corresponds to a group of continuous B mode images for a number of frames, that is to say an animated B mode image by sending and receiving signals in an “animated image mode” for continuous control of sending and receiving a signal as described above. At this time an ultrasonic wave echo signal for a number of frames is stored in the data storage unit 15 and at the same time is sent to the arithmetic processing unit 14.

When an ultrasonic wave velocity change image is required, the ultrasonic wave sending and receiving mechanism 11 controls the system so that signals are sent and received in the animated image mode before and after the application of heat to the measurement region, respectively, and thus, an ultrasonic wave echo signal for a number of frames before the application of heat that correspond to a group of B mode images (animated B mode image) before the application of heat, and an ultrasonic wave echo signal for a number of frames after the application of heat that correspond to a group of B mode images (animated B mode image) after the application of heat are respectively stored in the data storage unit 15.

Here, the ultrasonic wave sending and receiving mechanism 11 allows the number of scans per frame to be adjustable through an input operation in the input unit 18, and thus, it can be freely adjusted whether the frame rate is made slower instead of increasing the image quality by increasing the number of scans or the frame rate is made faster instead of allowing the image quality to deteriorate by reducing the number of scans. In addition, automatic adjustment in the “animated mode” increases the frame rate by thinning the number of scans unless the setting is changed through a manual operation.

The continuous wave power supplying mechanism 12 outputs ultrasonic continuous wave (for example, sine waves) having such a power as to be required when heat is applied to a measurement region of a subject from a high frequency power supply 12a and controls the probe 2 so that the oscillators S collectively send waves. The output voltage is approximately 10 V to 20 V, and a power supply dedicated for applying heat is used because a sufficient power for applying continuous waves is necessary. It is known that the depth of a portion in a living body to which heat can be applied is approximately 1/f where f is the frequency of the continuous waves. It is preferable for a portion at a depth of 5 cm or greater form the body surface to be able to be heated in order to diagnosis a fatty liver. The frequency band that makes the heating possible is 1 MHz to 3 MHz.

The switch unit 13 is provided between the oscillators S in the probe 2 and the ultrasonic wave sending and receiving mechanism 11 as well as in between the oscillator S and the continuous wave power supplying mechanism 12. The switch unit 13 is made of an electronic switch or a microrelay, and switches the connection to an oscillator S in the probe 2 between a terminal on the side where the ultrasonic wave sending and receiving mechanism 11 sends an ultrasonic pulse wave signal and receives an ultrasonic wave echo signal while scanning (terminal on the diagnostic side) and a terminal on the side where the continuous wave power supplying mechanism 12 allows the oscillators S to collectively send ultrasonic continuous waves (terminal on the heating side). As a result, the radiation of the subject with waves from one probe 2 can be switched between the radiation with continuous ultrasonic waves for the application of heat and the radiation with ultrasonic pulse waves for diagnosis.

Though the details are not described here, heat may be applied to a desired portion through convergence of an ultrasonic wave beam when a delay circuit is added to the circuit on the heating side, if necessary, so as to make focusing possible trough electronic means.

The arithmetic processing unit 14 uses the ultrasonic wave echo signal that has been acquired by the ultrasonic wave sending and receiving mechanism 11 or the ultrasonic wave echo signal that has been stored in the data storage unit 15 after being acquired so as to carry out an arithmetic process that is generally required for the process and analysis for the B mode image diagnosis and for the fat diagnosis. The arithmetic process in the present invention, can be divided into functional blocks for description, and is carried out in the process unit 21, the sampling unit 22, the ultrasonic wave velocity change analysis unit 23 and the fat region detecting unit 24.

The process unit 21 drives the ultrasonic wave sending and receiving mechanism 11 so that an ultrasonic wave echo signal for a number of scans that are required to form a group of B mode images for a number of frames within one period of the periodic change is continuously acquired form the measurement region that periodically changes so as to be stored in the data storage unit 15, and at the same time a process for forming a group of B mode images for a number of frames (animated B mode image) using the acquired ultrasonic wave echo signal. After the signal has been stored in the data storage unit 15, a process for forming a group of B mode images is carried out by reading the signal out form the unit 15. Here, “forming a group of B mode images” is to divide the ultrasonic wave echo signal for B mode images for a number of frames that have been continuously acquired into a number of ultrasonic wave echo signals for a number of scans for one frame so that the B mode images in the individual frames and the acquired ultrasonic wave echo signals are made to correspond with each other. That is to say, the “formation of an animated B mode image” is achieved here when the acquired ultrasonic wave echo signals for a number of frames and the locations of the scanning lines that form each B mode image can be made to correspond to each other. The thus formed group of B mode images is written into the image display control unit (DSC) 16.

In order to form an ultrasonic wave velocity change image, it is necessary to form two animated B mode images before and after the application of heat, and therefore, a process for acquiring an ultrasonic wave echo signal is carried out twice before and after the application of heat, and thus, the ultrasonic wave echo signal for a number of frames before the application of heat that correspond to the animated B mode image before the application of heat and the ultrasonic wave echo signal for a number of frames after the application of heat that correspond to the animated B mode image after the application of heat are stored in the data storage unit 15.

The sampling unit 22 calculates a two dimensional cross correlation from Formula (3) between the respective frames for the animated B mode image before (or after) the application if heat that has been formed in the process unit 21, and carries out a process for sampling one frame as the “standard image.”

The sampling unit 22 also calculates a two dimensional cross correlation from Formula (3) between the “standard image” and each frame of the animated B mode image after (or before) the application of heat, which is different from that used to sample the “standard image,” and carries out a process for sampling one frame as the “comparative image.”

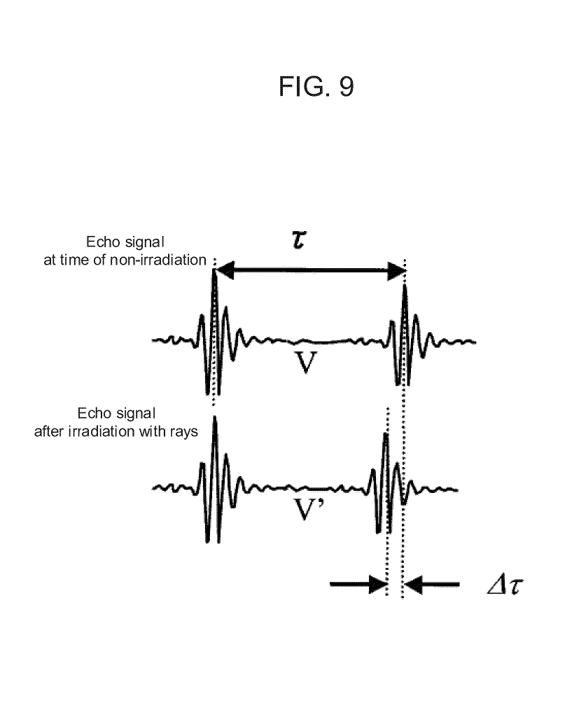

The ultrasonic wave velocity change analysis unit 23 calculates the waveform shift amounts (Δτ) in the ultrasonic wave echo signals before and after the application of heat for each section of part of the ultrasonic wave echo signal that corresponds to the sampled “standard image” and “comparative image” (ultrasonic wave echo signals for one frame stored in the data storage unit 15, respectively) in accordance with the same theory and method as in the prior art in FIG. 9, and in addition carries out process for calculating the pulse interval (τ) between the borders of the tissues within the measurement region. In addition, a process for calculating the ultrasonic wave velocity ratio in each portion (V′/V) from Formula (2) is carried out, and furthermore, an ultrasonic wave velocity change image is formed on the basis of the results of calculation of the ultrasonic wave velocity ratio so as to be written into the image display control unit (DSC) 16.

The fat region detecting unit 24 determines the portion of which the value of the calculated ultrasonic wave velocity ratio (V′/V) is smaller than one from among the respective portion as a fat region. In addition, the image of the fat region is differentiated from the other regions (for example, differently colored) so as to be written into the image display control unit (DSC) 16.

The image display control unit (DSC) 16 controls the system so that the image data that has been written into by the process unit 21, the ultrasonic wave velocity change analysis unit 23 and the fat region detecting unit 24 can be displayed as an image on the display unit 17 such as of a liquid crystal panel.

(Measurement Operation)

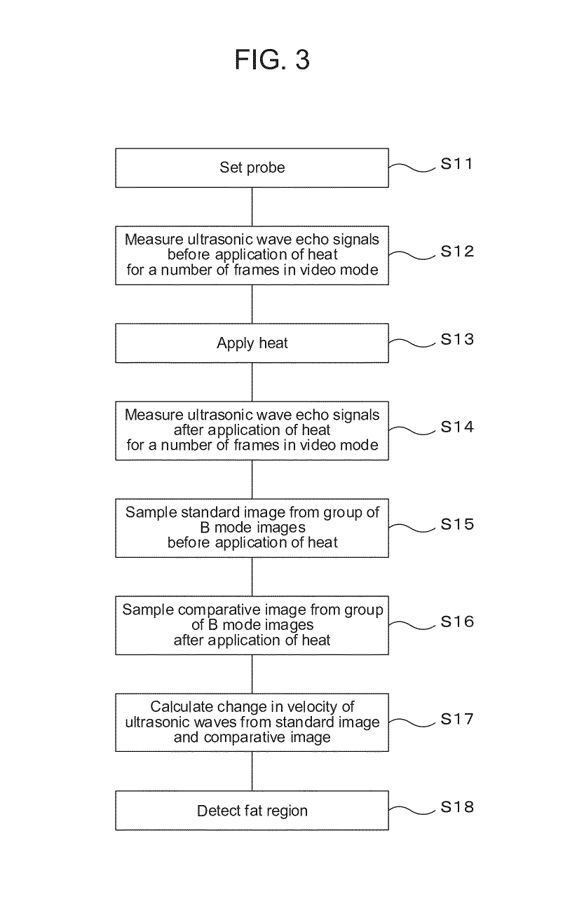

Next, the procedure of the operation for measuring the liver (fatty liver) in the body fat diagnostic apparatus 1 is described in reference to the flow chart in FIG. 3.

The switch unit 13 is switched to the “terminal on the diagnostic side” for the connection to the ultrasonic wave sending and receiving mechanism 11, and the oscillators S in the probe 2 are set so as to target the liver, which is the measurement region, through a gap between ribs of a subject (S11).

In addition, checking is carried out in advance in order to microscopically adjust the measurement position. At this time, the measurement region may be checked in a “video mode,” nonetheless it can be checked in the B mode image (still image) that has been taken without thinning the scanning because it is desirable to find the measurement position ultimately in clear image.

Next, the ultrasonic wave echo signal before the application of heat is measured in the “video mode” (S12). That is to say, ultrasonic pulse wave signals are repeatedly sent and at the same time ultrasonic wave echo signals that are reflected from the subject are received over a period of time that is longer than one period of the periodical change that is taking place in the subject so that ultrasonic wave echo signals for a number of frames within one period of the periodic change can be acquired.

At this time, the waves that have been sent and received may be scanned for each oscillator or may be scanned for a number of oscillators that are adjacent to each other so that the measurement regions can be concentrated in a location at a specific depth resulting form a so-called phase superposition. The acquired “ultrasonic wave echo signals before the application of heat” for a number of frames are stored and a group of B mode images (B mode video) is formed so as to be displayed on the display unit 17 through the image display control unit (DSC) 16.

Next, the probe 2 is kept set to the position where the ultrasonic wave echo signal before the application of heat was acquired, and the switch unit 13 is switch to the connection to the “terminal on the heating side” for the connection to the continuous wave power supplying mechanism 12. Thus, all of the oscillators emit ultrasonic continuous wave so as to apply heat to the measurement region until the temperature thereof rises by approximately 2° C. (S13).

Next, the ultrasonic wave echo signals after the application of heat are measured in the “video mode” after the measurement region has become stabilized in such a state as being heated (S14). That is to say, the application of heat is stopped, and the connection through the switch unit 13 is switched to the “terminal on the diagnosis side.” Thus, the same measurement conditions are provided as in S12 before the temperature decreases immediately after the application of heat has been stopped, and ultrasonic pulse wave signals are again repeatedly sent, at the same time ultrasonic wave echo signals that are reflected from the subject are received. Thus, the “ultrasonic wave echo signals after the application of heat” for a number of frames are stored, and a group of B mode images (B mode video) is formed so as to be displayed on the display unit 17.

Here, the operation can be carried out without failure and stabilized by incorporating a control sequence into the apparatus so that only an input operation to stop the application of heat can link a sequence of operations together from the sending of ultrasonic pulse wave signals immediately after the application of heat has been stopped and the reception of the ultrasonic wave echo signals.

Next, a standard image is sampled form the group of B mode images (B mode video) before the temperature changes (before the application of heat) (S15).

That is to say, the cross correlation R_{ZNCC} between the frames of B mode images included in the group of B mode images before the application of heat is calculated from Formula (3).

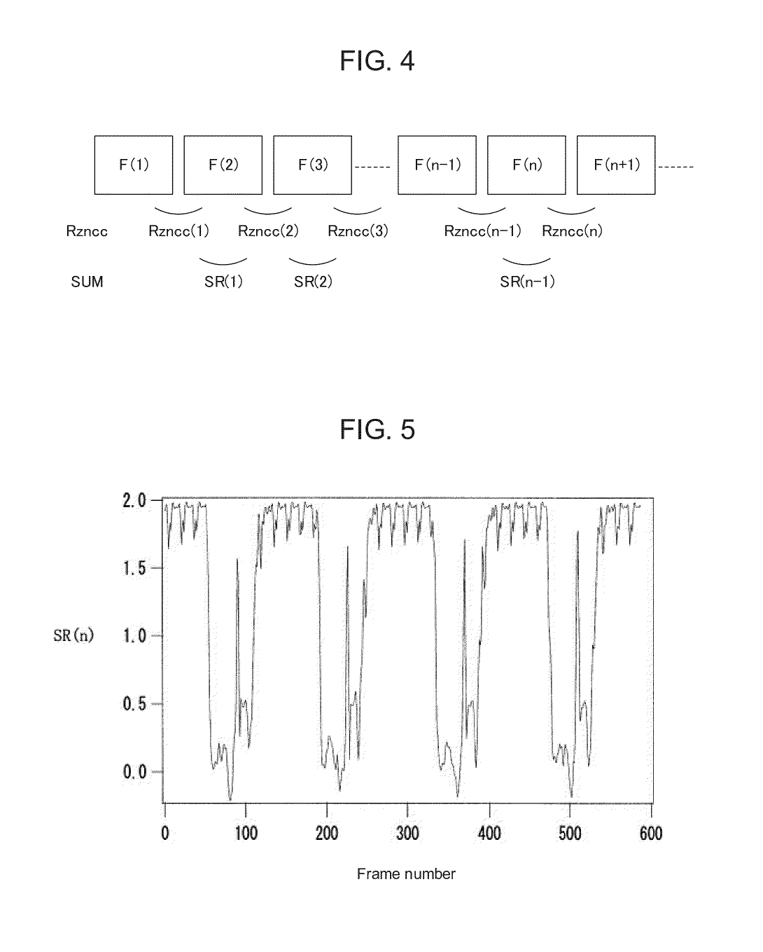

Typically, as shown in FIG. 4, the cross correlation R_{ZNCC}(1) between F(1) and F(2), the cross correlation R_{ZNCC}(2) between F(2) and F(3), . . . , the cross correlation R_{ZNCC}(n−1) between F(n−1) and F(n), the cross correlation R_{ZNCC}(n) between F(n) and F(n+1), . . . are sequentially calculated when F(1), F(2), . . . , F(n−1), F(n), F(n+1), . . . are B mode images included in the group of B mode images. As described above, R_{ZNCC} has such a value as to satisfy −1<R_{ZNCC}<1 where the closer to 1 the value is the higher the correlation the images have and the higher the similarity of the two images is. In other words the closer to 1 R_{ZNCC} is, the smaller the change between the two images is (the closer to a still image the image is). Therefore, the image of which the cross correlation R_{ZNCC} is closest to 1 is selected so that the image having the smallest change can be selected as the standard image.

Though the standard image may be selected using the above described cross correlation R_{ZNCC}, the sum SR(n) of two sequential cross correlations R_{ZNCC} in Formula (4) in the following may be calculated in the case where further stability between images is required.

SR(n)=R_{ZNCC}(n)+R_{ZNCC}(n+1) (4)

SR(n) has such a value as to satisfy −2<R_{ZNCC}<2, where the closer to 2 the value is the higher correlation the images have and the higher the similarity of the images in the three sequential frames is. In other words, the closer to 2 SR(n) is, the smaller the change between the three images is. Therefore, the sum of the cross correlations R_{ZNCC}, SR(n), that is closest to 2 is selected so that the image of which the change is smallest between the three sequential frames can be selected as the standard image.

FIG. 5 is a graph showing an example of calculation of the sum SR(n) of the cross correlations in an animal experiment using rabbits. In the graph, the lateral axis indicates the frame number of the group of sequential B mode images that are formed of ultrasonic wave echo signals before the application of heat and the vertical axis indicates the value of SR(n).

This graph includes approximately 4.5 periods of fluctuations having a large amplitude, on which fluctuations having smaller amplitudes and smaller periods superpose. The fluctuations having larger amplitudes are caused by respiration and the smaller fluctuations are caused by the heartbeat. B mode image for 600 frames are formed during approximately 4.5 periods of the periodical change.

The periods during which the value of SR(n) has risen close to 2 (excluding the effects of small amplitudes (heartbeat)) and sustains a value close to 2 for a while during fluctuation having a large amplitude represents a period from when the exhale of the breath begins until the exhale is complete from among the fluctuations having a large amplitude. The moment when the value of SR(n) that has risen to the peak of about 1.5 to 1.7 and then steeply fallen represents the moment when the inhale of the breath is complete.

This example shows that the image is most stable during the period before the exhale of the breath is complete and during the still period at the moment when the heartbeat has finished expansion or finished contraction where the value of SR has risen to close to 2.

Therefore, an actual image of which the value of SR is close to 2 can be selected so that an image where the state is almost still where the change of the image is the smallest can be sampled as “standard image” (hereinafter referred to as F(S)).

Next, a comparative image is sample from a group of B mode images (B mode video) after the temperature has changed (after the application of heat) (S16). That is to say, the cross correlation R_{ZNCC} between the B mode image in each frame included in the group of B mode images after the application of heat and the standard image F(s) is calculated from Formula (3).

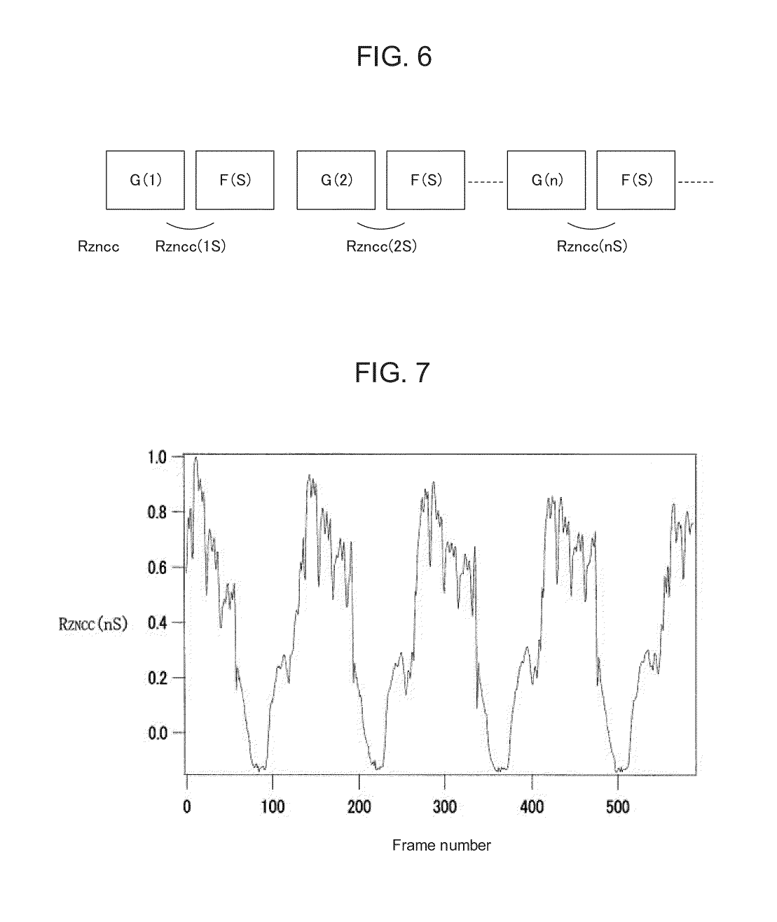

Typically, as shown in FIG. 6, the cross correlation R_{ZNCC}(1S) between G(1) and F(S), the cross correlation R_{ZNCC}(2S) between G(2) and F(S), . . . are sequentially calculated when G(1), G(2), . . . , G(n), . . . are B mode images included in the group of B mode images after the application of heat. In this case as well, the closer to 1 R_{ZNCC} is, the smaller the change between the two images is (similar images). Therefore, the image of which the cross correlation R_{ZNCC} is closest to 1 is selected so that the image similar to the standard image can be sampled as a “comparative image” (hereinafter referred to as G(S)).

FIG. 7 is a graph showing an example of calculation of the cross correlation R_{ZNCC}(nS) vis-à-vis the standard image in am animal experiment using rabbits. In this graph the lateral axis indicates the frame number of each of the sequential B mode images formed from ultrasonic wave echo signals after the application of heat and the vertical axis indicates the value of R_{ZNCC}(nS).

In this graph as well, the fluctuations having large amplitude are caused by respiration and smaller fluctuations are caused by the heartbeat. The value of R_{ZNCC}(nS) rises to the peak close to 0.8 to 1 for each period of the fluctuations having a large amplitude due to the respiration. The peak position fluctuates between 0.8 and 1 due to the effects of heartbeat and is closest to 1 when the respiration and the heartbeat are in sync with each other.

Therefore, a frame of which the value of R_{ZNCC}(nS) is at the peak close to 1 is selected so that an image in a state close to that of the standard image can be sampled as a “comparative image” (hereinafter referred to as G(S)).

Next, the ultrasonic wave echo signal that corresponds to the standard image F(S) and the ultrasonic wave echo signals that correspond to the comparative image G(S) are read out and the pulse interval (τ) and the amount of shift in the waveform (Δτ) are found, and thus, a change in the velocity of ultrasonic waves is calculated through the arithmetic operation on the basis of Formula (2) (S17). In addition, an ultrasonic wave velocity change image is prepared from the calculated Δ on the change in the velocity of ultrasonic waves so as to be displayed on the display unit.

Next, a region for which the ultrasonic wave velocity ratio (V′/V) is smaller than one is detected from the calculated data on the change in the velocity of the ultrasonic waves, and this region is determined to be a fat region. In addition, the data is written into the image display control unit (DSC) 16 so that the fat region can be displayed on the display unit (S18).

As a result of the above described operation, an image showing a fat region is displayed on the display unit 17, on the basis of which the results of fat diagnosis are displayed.

### Example

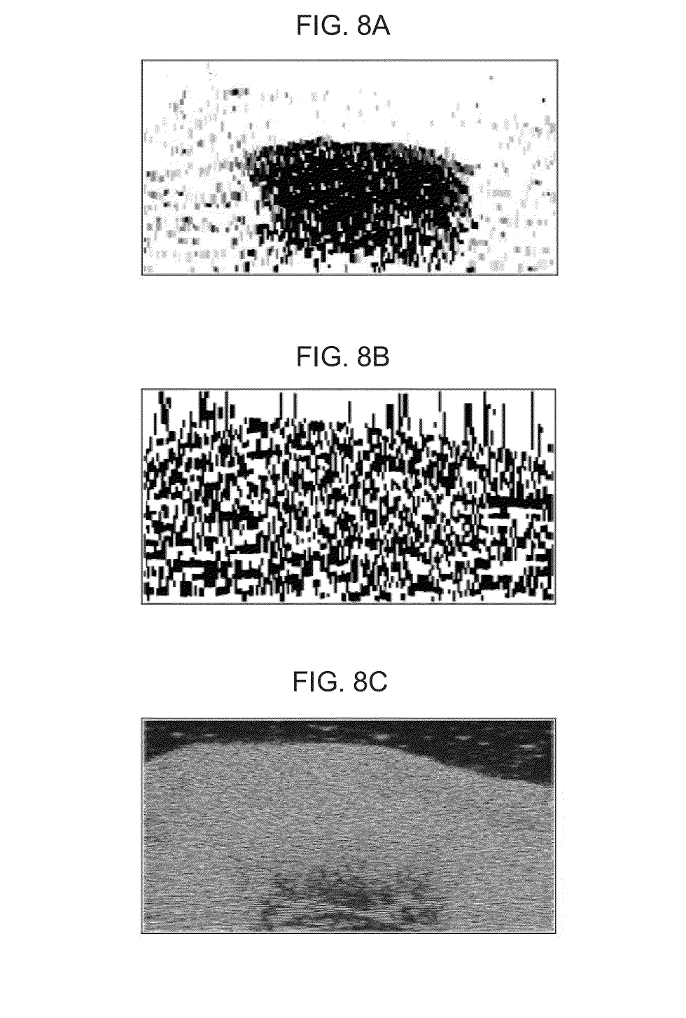

FIGS. 8A to 8C are images showing an example of the change in the velocity of ultrasonic waves where a fatty region has a distinctive color form the surrounding. FIG. 8A is an ultrasonic wave velocity change image that has been prepared by sampling the standard image F(S) and a comparative image G(S) in accordance with the above described procedure. Meanwhile FIG. 8B is an ultrasonic wave image that has been prepared from a frame sampled at random before and after the application of heat without taking the periodical change into consideration (in a state where the respiration and the heartbeat are out of sync). FIG. 8C is a B mode image that becomes the standard image F(S).

FIG. 8A clearly shows the fat region as a result of a sufficient synchronization (compensation for the movement) with the periodical change, while FIG. 8B only shows an unclear image.

Thus, according to the present invention, the effects of the periodical change due to respiration and heartbeat can be eliminated, and as a result fat diagnosis can be carried out using a clear image.

### Modifications

The present invention is not limited to the above described embodiments and various modifications are possible as long as the gist of the present invention is not deviated from.

For example, an echo signal is first acquired before the application of heat, after that heat is applied through ultrasonic waves, and then, an ultrasonic wave echo signal after the application of heat is acquired immediately after the application of heat has been stopped, according to the above described embodiments. Instead, heat is first applied to cause the desired temperature after the measurement position has been determined by checking a B mode image, then an ultrasonic wave echo signal after the application of heat is acquired immediately after the application of heat has been stopped, and subsequently, an ultrasonic wave echo signal at the time when no heat is applied, is acquired in a state where the temperature has returned to normal, and this maybe used as the echo signal before the application of heat. In doing so, the time and efforts required for the measurement increases. However, the blood vessels dilate so as to increase the blood flow in order to prevent the body temperature from rising when heat is applied. The increase in the blood flow makes the change in the temperature steep, and therefore, the change in the temperature per hour is greater in the measurement of the case where temperature decreases with time than the measurement of the case where the temperature rises with time, and therefore, a stable measurement becomes possible.

In addition, a standard image is selected from a group of B mode images before the temperature changes (before the application of heat), and a comparative image is selected from a group of B mode images after the temperature has changed (after the application of heat) according to the above described embodiments. However, the order of selecting the images can be switched.

Furthermore, the sum SR(n) of the cross correlations R_{ZNCC} of two terms in Formula (4) is used to sample the “standard image” according to the above described embodiments. However, the number of the used terms may be increased and the sum SR(n) of the cross correlations R_{ZNCC} of three or more terms may be used.

When the sum of the cross correlations of three terms is used, for example, an image having a small change between four sequential frames can be selected as the standard image.

Moreover, heat is applied using ultrasonic wave energy in accordance with above described embodiments. However, heat maybe applied in accordance with other methods such as irradiation with light, depending on the depth of the measurement region.

### INDUSTRIAL APPLICABILITY

The present invention can be applied to body fat diagnostic apparatuses for diagnosing fat in a living body having a periodical change.

### EXPLANATION OF SYMBOLS

1 body fat diagnostic apparatus
2 probe
3 control unit
11 ultrasonic wave sending and receiving mechanism
12 continuous wave power supplying mechanism
13 switch unit
14 arithmetic processing unit
15 data storage unit (memory)
16 image display control unit (DSC)
17 display unit (liquid crystal panel)
18 input unit (mouse, keyboard)
21 process unit
22 sampling unit
23 ultrasonic wave velocity change analyzing unit
24 fat region detecting unit

What is claimed is:

1. A body fat diagnostic apparatus for diagnosing fat by forming a B mode image on the basis of ultrasonic wave echo signals of which the number is the same as the scans for one frame acquired from a measurement region and by forming an ultrasonic wave velocity change image by calculating a change in the velocity of ultrasonic waves on the basis of ultrasonic wave echo signals of which the number is the same as the scans for one frame acquired from the measurement region before and after the application of heat, respectively, characterized by comprising:
a process unit for sequentially acquiring ultrasonic wave echo signals of which the number is the same as the scans required for forming a group of B mode images for a number of frames within one period of a periodical change from a measurement region that periodically changes so as to form said group of B mode images for a number of frames;
a data storage unit for storing the ultrasonic wave echo signals that correspond to the groups of B mode images for a number of frames that have been acquired before and after the application of heat, respectively;
a sampling unit for sampling one frame as a standard image by calculating a cross correlation that indicated the degree of change in the image between frames in one group of B mode images either before or after the application of heat, and for sampling one frame as a comparative image by calculating a cross correlation that indicated the degree of change in the image between said standard image and each frame in the other group of B mode images either after or before the application of heat;
an ultrasonic wave velocity change analyzing unit for forming an ultrasonic wave velocity change image by calculating a change in the velocity of ultrasonic waves on the basis of the ultrasonic wave echo signals of which the number is the same as scans that correspond to said standard image and said comparative image.

2. The body fat diagnostic apparatus according to claim 1, wherein
the number of scans per frame can be adjustable when a B mode image is formed, and
said process unit is formed so that images can be switched by thinning the scans per frame when said group of B mode images is formed and thereby increasing the number of frames that can be taken during one period of said periodical change.

3. The body fat diagnostic apparatus according to claim 1, wherein a zero-mean normalized cross correlation is used as said cross correlation.

4. The body fat diagnostic apparatus according to claim 2, wherein a zero-mean normalized cross correlation is used as said cross correlation.

5. The body fat diagnostic apparatus according to claim 3, wherein a sum of zero-mean normalized cross correlations is used as a cross correlation for sampling said standard image.

6. The body fat diagnostic apparatus according to claim 4, wherein a sum of zero-mean normalized cross correlations is used as a cross correlation for sampling said standard image.
